(19) United States
(12) Patent Application Publication
(10) Pub. No.: US 2021/0059788 A1
(43) Pub. Date: Mar. 4, 2021

(54) OVER-TRAVEL-FREE SYRINGE, IN PARTICULAR FOR ADMINISTERING PASTE-LIKE DENTAL COMPOSITIONS

(71) Applicant: KULZER GMBH, Hanau (DE)

(72) Inventors: Andrea KEMENY, Usingen (DE); Mike KONDZIELA, Weilburg (DE); Andreas UTTERODT, Neu-Anspach (DE); Hans Joachim ZUMKELLER, Weilrod (DE); Heiko FORTH, Wehrheim (DE); Kai LAUTENSCHLAGER, Alzenau (DE); Jörg KUPKA, Frankfurt (DE)

(73) Assignee: KULZER GMBH, Hanau (DE)

(21) Appl. No.: 16/956,762

(22) PCT Filed: Dec. 17, 2018
(86) PCT No.: PCT/EP2018/085274

§ 371 Date: Jun. 22, 2020

(30) Foreign Application Priority Data
Dec. 21, 2017 (DE) 10 2017 130 944.6

(51) Int. Cl. A61C 5/62; A61C 19/06; A61C 19/08; A61M 5/315

(52) U.S. Cl. CPC A61C 5/62; A61C 19/063; A61M 2202/048; A61M 5/31513; A61M 2205/0222; A61C 19/08

(57) ABSTRACT

Syringe (1) having a syringe body (5) and a proximal syringe outlet (11) in the syringe body (5) as well as having a syringe plunger (7) being axially (13) movable in the syringe body, having a proximal end and a distal end, wherein an elongate lumen (14) is formed inside the syringe plunger (7) coming from the proximal end (7a) of the syringe plunger (7) until the distal end (7b) of the syringe plunger, in which a movable inner plunger (8) having a proximal end (8a) and a distal end (8b) is arranged.

Syringe having a syringe body and a proximal syringe outlet in the syringe body as well as having a syringe plunger being axially movable in the syringe body, having a proximal end and a distal end, wherein an elongated lumen is formed inside the syringe plunger coming from the proximal end of the syringe plunger until the distal end of the syringe plunger, in which a movable inner plunger having a proximal end and a distal end is arranged.

There are reusable syringes in everyday medical practice, which are usually filled with a preparation, such as a dental composition, e.g. a fissure sealant, a filling material or an active agent, such as an anaesthetic. The application of pasty or liquid preparations, such as a fissure sealant, is often carried out using reusable syringes with the aid of cannulas. The cannulas are replaced each time. The cannulas are either attached to the syringe outlet or securely anchored via a Luer-Lock closure forming the syringe outlet.

The viscosities of the compositions may considerably vary from very solid, pluggable composites to so-called flow materials, which have a flowable consistency. Flowable dental compositions are usually put on the market in prefilled syringes and may usually be used several times. A syringe therefore contains an amount being sufficient for a variety of applications so that only the needles need to be replaced on the syringe.

High internal pressure builds up in the syringe body, synonymous with syringe cylinder, when pressing out the dental mass. After the user has applied a sufficient amount of the dental mass, the internal pressure in the syringe subsequently feeds further dental mass out of the syringe, although the user no longer exerts any pressure on the syringe. In case of commercially available syringes, the syringe body is siliconised to improve sliding behaviour between the syringe body and the syringe piston or plunger. This measure allows the syringe piston to be further carried out of the syringe body by the internal pressure in order to automatically release the internal pressure. However, siliconisation is not possible in all fields of application, as it can significantly impair the product properties of the dental mass to be applied. For example, the adhesion of the cured dental material in the tooth can be severely impaired by these lubricants or anti-adhesive agents. Therefore, this well-known siliconisation of the inner surface of the syringe body can severely impair the product properties of the dental mass. Thus, the friction between the inner surface of the syringe body and the syringe plunger is very high due to the lack of siliconisation of the syringe body. The high friction then prevents the syringe plunger or syringe piston from backing automatically after use, thus preventing setting of pressure compensation. The remaining high internal pressure in the syringe body then feeds the dental mass out of the syringe body through the cannula into the outer space. This behavior is called post-run and is undesirable for the user.

In its patent EP2016962B1, the competitor Voco describes a technical solution for this problem with a frictional connection element having a spring element and is so far the only manufacturer of dental materials whose syringes do not post-run.

In general, flowable and/or pasty compositions can also be dispensed from a syringe in other fields of application, such as soldering, application of pasty compositions, such as, for example, heat-conducting pastes, adhesives or sealing compounds or joint sealers, which can be present as 1K or 2K components.

It was the object of the invention to provide another syringe which avoids the above-mentioned post-run problems and preferably allows multi-use of syringes with compositions without post-run of the composition from the syringe. The post-run problem can lead to sticking of the composition to the underlay and possibly to an unintentional contamination of the composition. In addition, composition is discharged unnecessarily, which must be removed from the syringe outlet or an attached cannula before the syringe can be used again.

The object was solved by a non post-running syringe according to claim 1 as well by a kit according to claim 12 and also by a method according to claim 13 and the use according to claim 14.

A non-post-running syringe is understood to mean a syringe from which, after application of a liquid or pasty composition, in particular a pharmaceutical or dental composition, the composition does not escape from the syringe outlet without exertion of a force externally exerted to the syringe plunger and the inner movable plunger. The syringe plunger and the inner movable plunger are applied with an external force, in particular applied with a force by the user, during application of the composition.

According to an embodiment, a syringe is claimed in which friction (A) between the syringe plunger and the syringe body is greater than friction (B) between the inner plunger in the lumen, wherein friction (B) allows the inner plunger to be distally displaced without the syringe plunger being distally displaced in the syringe body, in particular when no proximally directed external force is exerted for inserting the inner plunger into the syringe body. The inner plunger acts as an internal pressure relief element.

Therefore, a syringe with the following functionalities is claimed:
applying a force to the syringe plunger and to the inner plunger to insert the syringe plunger and the inner plunger into the syringe body to discharge a composition stored in the syringe body in a defined manner from the syringe outlet or a cannula connected thereto,
stopping applying a force to the syringe plunger and the inner plunger, wherein the internal pressure build up in the syringe body, in particular of the composition, axially moves the inner plunger in the lumen of the syringe plunger to distal and the internal pressure in the syringe body is released, in particular to avoid post-run of the composition from the syringe outlet or the cannula at least in part.

The syringe according to the invention comprising a sleeve-like syringe body and a hollow syringe plunger avoids the post-run problem by means of an axially movable element being inside the syringe plunger or the piston in the piston base. Said element extends until the grip plate of the piston and is pushed to proximal together with the piston in the piston base when operating the syringe. The internal pressure in the syringe body pushes the movable element to distal after application thus reducing the internal pressure. For this purpose, the friction between the movable element and the piston with piston base is set lower than the friction between the syringe outlet, in particular through the cannula, and the dental mass. Post-run of the dental mass through the syringe outlet, in particular through the cannula to the outside, is thus avoided. When operating the syringe again, the user firstly pushes the movable element, in particular the inner plunger, to proximal until it is flush with the piston or the piston base. Since the sliding surfaces between the movable element and the piston with piston base do not come into contact with the dental mass, a lubricant such as silicone oil can also be used in this area to reduce friction.

A subject matter of the invention is a syringe having a syringe body, in particular the syringe body is formed in the form of a sleeve, the syringe body has a proximal syringe outlet as well as a syringe plunger being axially movable in the syringe body, having a proximal end and a distal end, an elongated lumen is formed inside the syringe plunger coming from the proximal end of the syringe plunger until the distal end of the syringe plunger, in which a movable inner plunger having a proximal end and a distal end is arranged. Preferably, the syringe body proximally has a syringe outlet which is formed as being a connector for fixing a cannula having a connector or an application pipe or extraction pipe, each having a connector.

In this context, it is preferred for the syringe piston being axially movable in the syringe body to be, at least in one region, in particular in one region to totally, with its outer surface, circumferentially in contact in a force-fit manner with the inner surface of the syringe body in the syringe body, which is present in the form of a sleeve preferably. Preferably, the proximal plunger of the syringe plunger and optionally the at least one sealing lip is circumferentially in contact in a force-fit manner with the inner surface of the syringe body. In this context, it is preferred for the sealing lip to be circumferentially arranged, in particular formed, particularly preferably glued on or welded on, on the outside of the outer periphery. In addition, it is preferred for an elongated lumen to be formed inside the syringe plunger coming from the proximal end of the syringe plunger until the distal end of the syringe plunger/piston, in which a movable inner piston having a proximal end and a distal end is arranged. Preferably, the elongated lumen being present inside the syringe plunger and running from the proximal end until the distal end of the syringe plunger is cylindrical in at least one to multiple sections. Particularly, preferably, the elongated lumen being present inside the syringe plunger and running from the proximal end until the distal end of the syringe plunger is continuously cylindrical. According to the invention, cylindrical is understood to mean a generalised right cylinder, i.e. all generatrices are perpendicular to a polygonal cross section, forming a prism. Particularly preferably, the lumen is present as right circular cylinder from the proximal to the distal end. Preferably, the inner surface of the lumen is flat, in particular smooth. Preferably, neither a protrusion and/or another step hinder movement of the inner plunger to distal. According to an embodiment, it is preferred if there is sealing element neither at the syringe plunger nor at the inner plunger at the proximal front face in the direction of the syringe outlet.

The outer surface of said inner plunger, at least in part to totally, and/or a circumferenting sealing lip on the outer surface of the inner plunger is in contact in a force-fit manner with the inner surface of the lumen of the syringe plunger.

Preferably, the force-fit between the outer surface of the inner plunger and inner surface of the lumen is smaller than the force-fit between the inner surface of the syringe body and the outer surface of the syringe plunger.

According to a particularly preferred embodiment in case of the syringe according to the invention, the operation force of the inner syringe plunger, in particular in the elongated lumen of the syringe piston, corresponds to less than 60% of the operation force of the syringe plunger, in particular in the syringe body. Particularly preferably, the operation force to be exerted of the inner syringe plunger amounts to 1 to 60% of the operation force to be exerted of the syringe plunger, preferably 20 to 60%, particularly preferably 30 to 60%. The operation force may also correspond to the press-out force from the syringe. The operation force may be measured using a Zwick-tensile and compression testing machine measuring the force to be exerted which is required to a) push forward the plunger or the piston in the syringe body (operation force of the syringe plunger) or to push the inner plunger in the lumen of the syringe body to proximal or distal, in particular to distal (operation force of the inner syringe plunger), wherein the plunger itself is fixed.

Furthermore, it is preferred for the inner plunger not to have any protrusions formed integrally on the inner plunger proximally at its outer periphery, but in particular the inner plunger may have a circumferenting sealing lip formed at the outer periphery. The material of the sealing lip may preferably comprise silicone.

In addition, it is preferred for the inner plunger and the syringe plunger to have an identical length or for the inner plunger to be shorter to of equal length than the syringe plunger. Particularly preferably, the proximal end of the inner plunger and the proximal plunger of the syringe plunger or the proximal piston of the syringe piston lay in a common plane in storage and/or handling position, in particular they form a common plane at their front face.

The proximal end, in particular the front face, of the inner plunger may be present pushed to distal into the lumen of the syringe plunger in the non-post-running position of the syringe after application of the composition. Therefore, in non-post-running position, the plane of the proximal end, in particular the front face, of the inner plunger may be displaced to distal in relation to the plane, in particular the front face, of the proximal plunger of the syringe plunger or the plane of the proximal piston of the syringe piston.

According to a preferred alternative, the inner plunger, at least in part or totally, on its outer surface and/or the inner surface of the lumen, at least in part or totally, is provided with at least one lubricant comprising a slip component comprising a lubricant or an additive or mixture of additives being present in the matrix and/or is superficially provided with an additive or a mixture of additive to reduce friction between the outer surface of the inner plunger and the inner surface of the lumen.

According to a preferred alternative, the inner plunger, at least in part or totally, on ist outer surface and/or the inner surface of the lumen, at least in part or totally, is provided with a lubricant to reduce friction between the outer surface of the inner plunger and the inner surface of the lumen.

According to an embodiment, the inner plunger does not extend into the proximal syringe outlet and/or the movable syringe plunger does not extend into the proximal syringe outlet.

According to a preferred embodiment, the inner plunger has a wing at the distal end. The syringe piston may also be a piston with proximal piston base.

According to an embodiment, an elongated lumen is formed inside the syringe plunger coming from the proximal end of the syringe plunger through the wing at the distal end of the syringe plunger, in which a movable inner plunger having a proximal end and a distal end with a wing is arranged.

The syringe body with inserted syringe plunger being axially movable preferably delimits a syringe body internal space having a volume to receive a liquid to pasty composition, in particular having a volume of 0.01 ml to 2 I, preferably of 0.01 ml to 500 ml, preferably of 0ö01 to 10 ml, particularly preferably having a volume of 0.01 ml to 2 ml. Particularly preferably, the syringe body with inserted syringe plunger being axially movable has a volume of 0.01 ml to 2 ml, in particular the volume amount to 0.2 ml to 1.5 ml, 0.5 go 1.2 ml are preferred, particularly preferably about 1 m. The syringe body preferably is present in the form of a sleeve, preferably in the form of a cylindrical sleeve provided with a syringe outlet on one side. The inner diameter of the syringe body in the region where the axially movable syringe plunger is situated, preferably amounts to 4 to 14 mm, preferably 5 to 8 mm. The outer diameter of the cannula preferably amounts to 0.4 to 1.4 mm/27 G to 17 G), preferably 0.6 to 1.2 mm (23 G to 18 G), particularly preferably 0.9 mm (20 G). G is the gauge measure and corresponds to the number of drawing steps of the tube blank. The cannula is made of a metal preferably.

Furthermore it is preferred for the elongated lumen to be axially, in particularly axially along the entire longitudinal axis of the syringe plunger, formed in the syringe plunger from the proximal to the distal end. Preferably, the syringe plunger is rotationally symmetric, preferably is comprises at least one rotationally symmetric syringe plunger. Moreover, the elongated lumen is rotationally symmetric preferably. The syringe piston is defined as the syringe plunger without distal wings which the syringe may have additionally. Syringe plunger and syringe piston may therefore be identical, in particular if the distal wings are optionally provided at the syringe plunger. A syringe plunger according to the invention may preferably be circular cylindrical with a lumen according to the invention. Thus, an elongated lumen is formed inside the syringe plunger coming from the proximal end of the syringe plunger until the distal end of the syringe plunger, since, in particular, the syringe piston is identical the syringe plunger without wing and is a part of the syringe plunger with wings.

In addition, the syringe plunger a) may have at least one plunger at the proximal end and, optionally, at least one sealing lip in the proximal region of the syringe plunger, and a wing at the distal end, or b) the syringe plunger may be present in the form of a piston having, optionally, a plunger at the proximal end and, optionally, at least one circumferenting sealing lip in the proximal region of the syringe plunger and, optionally, a wing at the distal end.

In further embodiments, the syringe plunger a) may have at least one plunger at the proximal end and, optionally, at least one sealing lip circumferenting at the outer periphery or an O-ring being arranged at the outer periphery, and a wing at the distal end, or b) the syringe plunger may be present in the form of a piston having, optionally, a plunger at the proximal end and, optionally, at least one sealing lip circumferenting at the outer periphery or an O-ring being arranged at the outer periphery, and, optionally, a wing at the distal end. Preferably, the inner plunger, at least in part, in particular partially to totally, on its outer surface, and/or at least in part, in particular partially to totally, or the sealing lip, and/or the O-ring has a lubricant, and/or the inner surface of the lumen, at least in part, has a lubricant. The lubricant may be a fluid substance or a fluid composition, such as silicone oil. The lubricant reduces the friction between the outer surface of the inner plunger and the inner surface of the lumen. The lubricant is also considered an additive.

Furthermore, it is preferred for at least one continuous elongated lumen to run through the entire syringe plunger inside the syringe plunger coming from the proximal end of the syringe plunger, in particular from the proximal surface, until the distal of the syringe plunger, in particular until the distal surface of the syringe plunger, and wherein a movable inner plunger having a proximal end and a distal end, in particular having a wing at the distal end, is arranged in the lumen. Preferably, the lumen and the inner plunger are axially arranged in the syringe plunger, in particular they are movably arranged against each other. In particular, the lumen runs through the plunger of the syringe plunger until the wing, preferably the lumen is axially arranged in the syringe plunger and a movable inner plunger having a proximal end and a distal end, in particular having a wing at the distal end, is arranged in the elongated axial lumen, wherein the inner plunger is axially movable. Preferably, the syringe plunger and the inner plunger are of equal length so that in particular the proximal and the distal end of the syringe plunger and of the inner plunger end in the same plane. Preferably, the syringe plunger has only one elongated lumen to receive an inner plunger.

Preferably, the force-fit between the outer surface of the inner plunger and the inner surface of the lumen is smaller than the force-fit between the inner surface of the syringe body and the outer surface of the syringe plunger.

The syringe outlet at the syringe body may be formed as a connector or a part of a connector system. The following are basically considered as connector or connector systems: plug-in connection, detent, rotary connection, rotary/detent connection, any Luer systems, such as a Luer lock system, a rotating Luer lock system, plug-in system comprising two hollow cones, screw plug, or a plug-in system having a cam, a bayonet catch. A connector may be formed as a female Luer lock connector on the inside and as a male Luer lock connector on the outside. The female Luer lock connector may be fixed at the male Luer lock connector of the syringe outlet of the syringe body. A further connector, e.g. a cannula, may be formed as a female Luer lock connector to be connected to the male Luer lock connector of the afore-mentioned connector. The syringe or the syringe outlet, in particular in the form of a connector, each independently, may have a protective cap preferably having a connector. A syringe may be provided with a connector, preferably made of plastic, and with a cannula, in particular made of metal. Therefore, the kit may also comprise such a connector with a cannula.

Within the scope of the invention, connectable is understood to mean a connection of two of the afore-mentioned parts, being tightly sealed against escape of liquid and the connection of which is detachable.

According to a particularly preferred embodiment, a cannula optionally having a connector or having a part matching the part of the connector system is arranged at the proximal syringe outlet, at the connector or at the part of a connector system. The proximal syringe outlet is preferably present as connector or part of a connector system.

Furthermore it is preferred for friction (A) between the syringe plunger and the syringe body to be greater than friction (B) of the inner plunger in the lumen of the syringe plunger, in particular friction (A) is 10% greater than friction (B), preferably friction (A) is 50% greater than friction (B).

According to an alternative embodiment, the syringe body, the syringe plunger and/or the inner plunger, each independently, may be made of a thermoplastic plastic, such as TPE (thermoplastic elastomers). Preferred thermoplastic plastics comprise: PE (polyethylene), PP (polypropylene), polyacetals, in particular POM (polyoxymethylene), PBT (Polybutylene terephthalate), PET (polyethylene terephthalate), PETP (polyethylene terephthalate), PA (polyamide) and/or PTFE (polytetrafluoroethylene). Likewise, the plastics may be provided in the matrix or superficially with at least one additive or a mixture of additives, preferably so-called lubricants, to reduce the friction, such as, for example: oleic acid amide(s)/oleamide(s), erucamide ((Z)-docos-13-enamide), ethylene bis-oleamide and/or stearyl erucamide ((Z)-N-octadecyldocos-13-enamide), solid lubricants, such as polytetrafluoroethylene (PTFE), molybdenum disulfide (MoS), boron nitride (BN), silicone oil or also further oily to waxy substances which may be compounded into the polymer matrix. PTFEE is only compounded into the plastic in the matrix as a lubricant if the matrix is not already made of this plastic. Alternatively, PTFE may be applied onto a core of another thermoplastic plastic as a superficial plastic layer. Particularly preferably, the inner plunger and/or the syringe plunger/piston, each independently, may be made of a plastic being provided in the matrix or superficially with at least one additive or a mixture of additives, preferably the additive(s) is or are so-called lubricants. Preferably, the proximal end of the syringe plunger/piston and/or the proximal end of the inner plunger are superficially not provided with additives. Likewise, the syringe body may preferably not comprise the additive.

Optionally, the two proximal ends of the syringe plunger/piston and the inner plunger may be provided with an addition film without additives so that preferably the liquid to pasty composition do not come into contact with additives. If the at least one additive is present in the matric, it may be preferred in an alternative for the additive to migrate from the matrix to the surface additionally. A particularly preferred plastic for the syringe body, plunger and/or inner plunger may be a two-phase plastic component which preferably has reduced friction on the surface and in the core consist of a plastic having increase mechanical properties compared the plastic used on the surface. Plastic components having a polyacetal core, such as POM, with an optional PTFE surface layer optionally filled with inorganic particles are examples for appropriately constructed syringe bodies and/or syringe plungers and/or inner plungers.

According to a further alternative embodiment, a) the distal end of the syringe plunger may have a wing made of a thermoplastic plastic, and/or b) the proximal end of the syringe plunger may have, proximally and/or in the proximal region at the outer periphery, a circumferential sealing lip or an O-ring, in particular the sealing lip or the O-ring is made of a thermoplastic and/or elastic plastic. Preferred plastics comprise a thermoplastic plastic, an elastomer or thermoplastic elastomer. Preferably, the sealing lip or the O-ring is made of a silicone. In this context, it is preferred for the sealing lip to be an integral part of the syringe plunger. The O-ring may be attached as a separate component to the syringe plunger, preferably in a circumferential groove.

In order to fix the inner plunger to the syringe plunger, in particular up to a defined force, during storage or to assist in discharging a composition from the syringe, a) the wing of the inner plunger may have a lateral cam at the distal end, being engaged into a recess in the wing of the syringe plunger inside the distal end in the lumen, in particular in the state of storing the syringe, or b) a lateral cam is provide at the distal end inside the lumen of the wing of the syringe plunger, being engaged or which may be engaged into a recess in the wing of the inner plunger outside at the distal end, in particular in the state of storing, or c) the syringe plunger may be formed as being at least one piston having at least one lateral cam at the distal end inside the lumen, being engaged or which may be engaged into a recess at the outside of the inner plunger at the distal end, in particular in the state of storing, or d) the inner plunger may be formed as being at least one piston having at least one cam on the outside, being engaged or which may be engaged into a recess in the syringe piston inside the lumen, in particular in the state of storing. The cam and the corresponding recess or the corresponding recesses may be arranged at the syringe plunger and inner plunger at any position.

Likewise a subject matter of the invention is kit comprising a syringe, wherein the syringe is filled with a fluid or pasty pharmaceutical or dental composition, wherein the dental composition comprises at least one monomer. Preferably, the syringe is filled with a dental fissure sealant, a gingiva retraction paste astringent, dental glue, self-etching adhesive, tissue glue, in particular cyanoacrylate tissue glue, gluten protein solution, thrombin solution with calcium chloride, pharmaceutical active agent, such as an anaesthetic or antibiotic.

The liquid or pasty pharmaceutical or dental compositions preferably have a viscosity in the range of 12700 to 64000 mPa·s, preferably in the range of 12700 to 40000 mPa·s. Uncoloured pastes may have a viscosity of up to 60000 mPa·s. The viscosity may be determined according to DIN EN ISO 3219_1994-10, DIN 53019-1_2008-09 or DIN 1342 1_2003-11. The testing may preferably be performed with the following testing parameters: load viscosity: shear rate d(gamma)dt=0.1 . . . 50 1/s linear, time=30 measuring points, measuring point length=6 seconds. Preferably, the measurement may still be performed at 20 to 23° C., preferably at 23° C., and preferably with a rheometer such as Anton Paar-Physica MCR 301.

Furthermore, a subject matter of the invention is a method for substantially non-post-running application of fluid to pasty compositions from a syringe having a syringe body and a proximal syringe outlet, in particular in the form of a connector and preferably with a cannula attached via a connector, in the syringe body as well as having a syringe plunger being axially movable in the syringe body, having a proximal end and a distal end, wherein an elongated lumen is formed inside the syringe plunger (coming from the proximal end of the syringe plunger until the distal end of the syringe plunger, in which a movable inner plunger having a proximal end and a distal end is arranged, comprising the steps of:
providing the syringe with the fluid to pasty composition in the volume formed in the syringe body with inserted syringe plunger being axially movable, wherein an elongated lumen is formed inside the syringe plunger coming from the proximal end of the syringe plunger until the distal end of the syringe plunger, in which a movable inner plunger having a proximal end and a distal end is arranged, wherein, in particular, a part of the distal end of the inner plunger and a part of the distal end of the syringe plunger lay in a planar plane,
applying a force to the syringe plunger and to the inner plunger to insert the syringe plunger and the inner plunger into the syringe body to discharge the composition in a defined manner from the syringe outlet or a cannula connected thereto,
stopping applying a force to the syringe plunger and the inner plunger, wherein the internal pressure build up in the syringe body axially, in particular relative to the syringe plunger, moves the inner plunger in the lumen of the syringe plunger to distal and the internal pressure in the syringe body is released, in particular to avoid post-run of the composition from the syringe outlet or the cannula at least in part.

On the one hand, the construction of the syringe according to the invention allows the user to recognize when the syringe has released after use by the distal end of the movable inner plunger protruding from the distal end of the syringe plunger. In addition, another particular advantage of the inner plunger is that the user is able to carry out fine dosing during application by applying only the protruding distal end of the inner plunger again. The syringe plunger preferably is axially movable along the longitudinal axis of the syringe piston and/or the inner plunger.

The press-out force of a composition from the syringe at bent/angled cannula, in particular with an angle in the range of 30 to 54°, and an outer diameter of 0.9 mm (20 G) may preferably amount to 30 to 150 N.

Furthermore, a subject matter of the invention is use of the syringe for non-post-running application of fluid or pasty pharmaceutical or dental compositions, such as, in particular, a dental fissure sealants, gingiva retraction pastes, astringents, dental glues, self-etching adhesives, tissue glues, in particular cyanoacrylate skin glues, gluten protein solutions, thrombin solutions with calcium chloride, with pharmaceutical active agents, such as an anaesthetic or antibiotic.

Non-post-running application is understood to mean that less than 0.5 mg, preferably less than 0.4 mg per dispensing operation escapes from the syringe.

The following figures clarify the invention in more detail without limiting the invention to the exemplary embodiments.

The figures show:

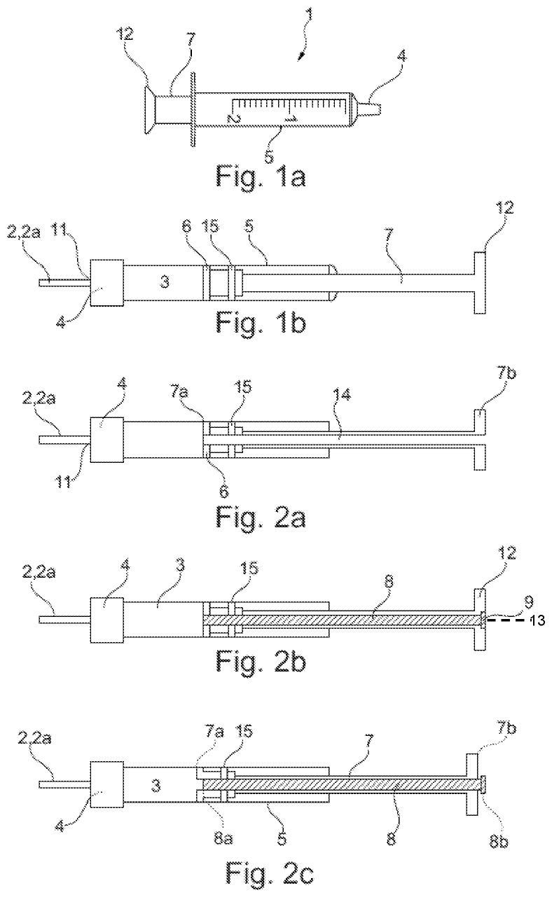

FIG. 1a, 1b: syringes 1 with syringe body 5 and syringe plunger 7 with syringe outlet 11 in the form of a connector 4 according to the state of the art

FIG. 2a, 2b, 2c: syringe 1 according to the invention with syringe outlet 11 in the form of a connector 4 or a connector system 4a with syringe plunger 7 with lumen 14 and inner plunger 8

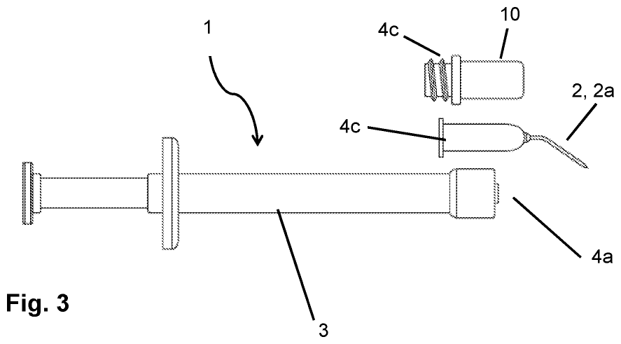

FIG. 3: syringe 1 with cannula 2a and closure 10

FIGS. 1a and 1b, disclose syringes 1 according to the state of the art. The syringes 1 have a syringe body 5 with syringe plunger 7 or piston with piston base 7 with distal wing 12. At the syringe in FIGS. 1a and 2a, connector 4 is shown as being a Luer lock connector. In FIG. 1b, syringe body 5 is shown as being a sleeve with syringe outlet 11 as being a connector 4 with attachment 2 in the form of a cannula 2a. Composition 3 is filled-in in syringe body 5. Proximal plunger 6 possibly with sealing lip 15 or proximal piston base 6 together with syringe plunger 7 and distal wing 12 serves for pressing out the composition 3. Since composition 3 is to be discharged through a cannula having a very small diameter, high internal pressure is build up inside syringe body 5 leading to post-run of the composition from the cannula after application, although no more force is exerted to syringe plunger 7.

FIGS. 2a, 2b and 2c, show syringes 1 according to the invention having a syringe body 5 and a syringe outlet 11 in the geometry of a connector 4 with attached attachment 2, here a cannula 2a with an own connector. A pasty composition 3 is stored in the syringe body 5, which is to be discharged from the cannula 2a having a very small inner diameter by means of the syringe plunger 7. Syringe plunger 7 may have a proximal plunger 6 in the proximal region at proximal end 7a as well as a sealing lip 15 in the proximal region. The user jointly exerts a force on both wings 9 and 12 at distal end 8b of the inner plunger 8 and distal end 7b of the syringe plunger 7, which is sufficient to discharge an amount of the composition for application from the cannula. After application, the user may lay syringe 1 down. Due to the internal pressure build up in the syringe body 5, the inner plunger 8 can axially be moved inside the syringe plunger 7 to distal, in particular along the longitudinal axis 13 of the syringe plunger and of the inner plunger. In so doing, the pressure is released and the composition no longer flows out of the cannula, but can relieve inside the syringe body. In this position, proximal end 7a of syringe body 7 lays before proximal end 8a of inner plunger 8, as shown in FIG. 2c. If necessary, the user may now insert distal end 8b of inner plunger 8 back into syringe plunger 7 for fine application. FIG. 3 shows a syringe 1 with syringe body 3 and a connector 4a to receive a closure 10 with connector 4c as well as an attachment 2 with cannula 2a and connector 4c.

### LIST OF REFERENCE NUMERALS

1 syringe
2 attachment, in particular cannula 2a
3 composition in syringe body
4 connector, or part of a connector system 4a, connector 4b, 4c member-matching

member 4a of the connector system, connector at the proximal syringe body having an internal or external thread at the proximal end
5 syringe body
6 proximal plunger of the syringe plunger/piston of the syringe piston
7 syringe piston/syringe plunger, piston with piston base, 7a proximal end of the syringe plunger/piston, 7b distal end of the syringe plunger/piston,
8 inner plunger, in particular having a distal wing, 8a proximal end of the inner plunger, 8b distal end of the plunger
9 distal wing of the inner plunger
10 closure
11 syringe outlet
12 distal wing of the syringe piston/syringe plunger
13 longitudinal axis
14 elongated lumen
15 sealing lip, O-ring

1. A syringe (1) having a syringe body (5) and a proximal syringe outlet (11) in the syringe body (5) as well as having a syringe plunger (7) being axially (13) movable in the syringe body, having a proximal end and a distal end, an elongated lumen (14) being formed inside the syringe plunger (7) coming from the proximal end (7a) of the syringe plunger (7) until the distal end (7b) of the syringe plunger, in which a movable inner plunger (8) having a proximal end (8a) and a distal end (8b) is arranged, wherein in that the inner plunger (8), at least in part, on its outer surface, and/or the inner surface of the lumen (14), at least in part, is provided with at least one lubricant so that the force-fit between the outer surface of the inner plunger (8) and the inner surface of the lumen (14) is smaller than the force-fit between the inner surface of the syringe body (5) and the outer surface of the syringe plunger (7).

2. The syringe (1) according to claim 1, wherein
the elongated lumen (14) is axially (13) formed in the syringe plunger (7) from the proximal (7a) to the distal end (7b) in the rotationally symmetric syringe plunger, the elongated lumen being formed rotationally symmetrically.

3. The syringe (1) according to claim 1,
wherein the proximal syringe outlet (11) in the syringe body (5) is a connector (4) or a part of a connector system (4a).

4. The syringe (1) according to claim 1, wherein
a) the syringe plunger (7) has at least one plunger (6) at the proximal end and, at least one sealing lip (15) circumferenting at the outer periphery or an O-ring being arranged at the outer periphery in the proximal region of the syringe plunger (7), and a wing (12) at the distal end, or
b) the syringe plunger (7) is present in the form of a piston having, a plunger (6) at the proximal end and, at least one sealing lip (15) circumferenting at the outer periphery or an O-ring being arranged at the outer periphery in the proximal region of the syringe plunger (7) and a wing (12) at the distal end.

5. The syringe (1) according to claim 4, wherein
i) the sealing lip (15) and/or the O-ring (15) is provided with at least one lubricant, and/or
ii) at least one of the following components comprising the inner plunger (8), sealing lip (15), O-ring (15) and/or the syringe plunger (7), each independently, are made of at least one plastic being provided with at least one additive or a mixture of additives in the matrix, and/or
iii) at least one of the following components comprising the inner plunger (8), sealing lip (15), O-ring (15) and/or the syringe plunger (7), each independently, is superficially provided with at least one additive or a mixture of additives
to reduce friction between the outer surface of the inner plunger (8) and the inner surface of the lumen (14).

6. The syringe (1) according to claim 4, wherein
i) the syringe body (5), the syringe plunger (7), the sealing lip (15), the O-ring (15) and/or the inner plunger (8), each independently, are made of at least one thermoplastic plastic, and/or
ii) the syringe plunger and/or the inner plunger, each independently, are made of at least one thermoplastic plastic.

7. The syringe (1) according to claim 1, wherein
at least one continuous elongated lumen (14) runs inside the syringe plunger (7) coming from the proximal end (7a) (surface) until the distal end (7b) (surface) of the syringe plunger through the whole syringe plunger (7), in which a movable inner plunger (8) having a proximal end (8a) and a distal end (8b), in particular having a wing (9), is arranged.

8. The syringe (1) according to claim 1, wherein
a cannula (2a), having a connector (4b) or having a part (4) matching at the part (4a) of the connector system, is arranged at the proximal syringe outlet (11), at the connector (4) or at the part of a connector system (2a).

9. The syringe (1) according to claim 1, wherein
the syringe body (7) with the inserted syringe plunger (7) being axially movable has a volume of 0.01 ml to 2 ml, in particular or the volume amounts to 0.2 ml to 1.5 ml.

10. The syringe (1) according to claim 1, wherein
the friction (A) between the syringe plunger (7) and the syringe body (5) is greater than the friction (B) of the inner plunger (8) in the lumen (14) of the syringe plunger (7), friction (A) being 10% greater than friction (B), or friction (A) being 50% greater than friction (B).

11. The syringe (1) according to claim 1, wherein
a) the distal end (7b) of the syringe plunger (7) has a wing (12) made of thermoplastic plastic, and/or
b) the proximal end (7a) of the syringe plunger (7) has, proximally and/or in the proximal region at the outer periphery, a circumferential sealing lip or an O-ring, in particular the sealing lip or the O-ring isbeing made of a thermoplastic plastic, elastomer or a thermoplastic elastomer.

12. The syringe (1) according to claim 11, wherein
a) the wing (9) of the inner plunger (8) has a lateral cam at the distal end (8b), being engaged into a recess in the wing (12) of the syringe plunger (7) inside the distal end in the lumen (14), or
b) a lateral cam is provide at the distal end (7b) inside the lumen of the wing (12) of the syringe plunger (7), being engaged into a recess in the wing (9) of the inner plunger (8) outside at the distal end (8b), in the state of storing the syringe, or
c) the syringe plunger (7) is formed as being at least one piston having at least one lateral cam at the distal end (7b) inside the lumen (14), being engaged into a recess at the outside of the inner plunger (8) at the distal end (8b), in the state of storing the syringe, or
d) the inner plunger (8) is formed as at least one piston having at least one cam on the outside, being engaged into a recess in the syringe piston inside the lumen (14), in the state of storing.

13. A kit comprising a syringe (1) according to claim 1, wherein the syringe is filled with a fluid or pasty pharmaceutical or dental composition, with a dental fissure sealant, gingiva retraction past, astringent, a dental glue, a self-etching adhesive, a tissue glue, with a cyanoacrylate skin glue, gluten protein solution, thrombin solution with calcium chloride, pharmaceutical active agent, optionally an anaesthetic or antibiotic.

14. (canceled)

15. (canceled)

16. The syringe (1) according to claim 6, wherein
i) the syringe body (5), the syringe plunger (7), the sealing lip (15), the O-ring (15) and/or the inner plunger (8), each independently, are made of at least one thermoplastic plastic, comprising thermoplastic elastomers, polyethylene, polypropylene, polyacetal, optionally POM, PBT, PET, PETP, PA and/or PTFE, and/or
ii) the syringe plunger and/or the inner plunger, each independently, are made of at least one thermoplastic plastic, comprising thermoplastic elastomers, polyethylene, polypropylene, polyacetal, optionally POM, PBT, PET, PETP, PA and/or PTFE, and the plastic comprising at least one additive or a mixture of additives in the matrix, at least one lubricant or a mixture of lubricants to reduce the friction, at least one oleamide, erucamide ((Z)-docos-13-enamide), ethylene bis-oleamide and/or stearyl erucamide ((Z)-N-octadecyldocos-13-enamide), solid lubricants, optionally polytetrafluoroethylene (PTFE), molybdenum disulfide (MoS), boron nitride (BN), silicone oil or further oily to waxy substances, and/or
iii) the syringe plunger and/or the inner plunger, each independently, are made of at least one thermoplastic plastic, comprising thermoplastic elastomers, polyethylene, polypropylene, polyacetal, such as POM, PBT, PET, PETP, PA and/or PTFE, and the plastic is superficially provided with at least one additive or a mixture of additives, at least one lubricant or a mixture of lubricants to reduce the friction, with at least one oleamide, erucamide ((Z)-docos-13-enamide), ethylene bis-oleamide and/or stearyl erucamide ((Z)-N-octadecyldocos-13-enamide), solid lubricants, optionally polytetrafluoroethylene (PTFE), molybdenum disulfide (MoS), boron nitride (BN), silicone oil or further oily to waxy substances.
